(19) United States
(12) Patent Application Publication
(10) Pub. No.: US 2005/0054653 A1
(43) Pub. Date: Mar. 10, 2005

(54) Pteridine derivatives, method of producing them and their application

(75) Inventors: Gerhard Eisenbrand, Heidelberg (DE); Karl-Heinz Merz, Bad Durkheim (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: Faustus Forschungs Cie. Translational Cancer Research GmbH

(21) Appl. No.: 10/896,659

(22) Filed: Jul. 22, 2004

Related U.S. Application Data
(63) Continuation of application No. PCT/EP03/00676, filed on Jan. 23, 2003.

(30) Foreign Application Priority Data
Jan. 23, 2002 (DE) 102 02 468.5

(51) Int. Cl. A61K 31/525; C07D 475/02

(52) U.S. Cl. 514/251; 544/260

(57) ABSTRACT

Compounds of the general formula (I) and their acid addition salts are provided 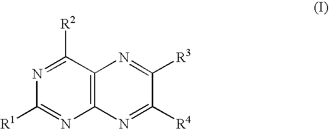 wherein:

R¹ signifies a piperazino, p-phenylenediamino, a 2,5-diazabicyclo-[2.2.1]-heptane, a 2,5-diazabicyclo-[2.2.2]-octane radical or a 3,8-diazabicyclo-[3.2.1]-octane radical, which in each case can be substituted with at least one substituent,

R², R⁴, which are in each case the same, signify a pyrrolidino, thiazolidino, oxazolidino or imidazolidino radical, which in each case can be substituted with at least one substituent, and

R³ signifies an alkyl, alkoxy, alkylmercapto or an alkylamino radical, which in each case can be substituted with at least one substituent. These pteridine derivatives are suitable for the inhibition of phosphodiesterases and therefore for the prophylaxis and/or treatment of thrombo-embolic, neurodegenerative diseases, inflammatory diseases, asthmatic diseases as well as hemato-oncological diseases.

### CROSS-REFERENCE TO RELATED APPLICATIONS

This application is a continuation of International Application No. PCT/EP03/00676, filed Jan. 23, 2003, the disclosure of which is incorporated herein by reference.

### BACKGROUND OF THE INVENTION

This invention relates to new pteridine derivatives and to methods of their production. Furthermore, this invention relates to the use of these pteridine derivatives, including for the inhibition of cAMP-specific phosphodiesterases, for the inhibition of tumor growth, for the prophylaxis of thrombo-embolic diseases and for the treatment of inflammatory, neurodegenerative diseases and asthma.

Merz et al. have already described in Journal of Medicinal Chemistry, 41:4733-4743 (1988) the production of 7-benzylamino-6-chloro-2-piperazino-4-pyrrolidinopteridine and derivatives thereof which are free of positional isomers. It has been shown that the produced compounds can be used as inhibitors of the cyclic nucleotide phosphodiesterases (PDEs) and can inhibit the growth of tumor cells. With the 6-chloro-substituted pteridines it has been shown that for a high activity of the heterocyclic substituent in the 2-position of the pteridine ring system a basic nitrogen should be included in the 4′-position, as shown by piperazine.

In German published patent application DE 3540952, 2-piperazino-pteridines are described which are substituted in the 6-position by a halogen atom, selected from a fluorine, chlorine or bromine atom. It has been shown that these compounds can inhibit the PDE activity of tumor cells and of human thrombocytes in vitro.

German published patent application DE 3323932 also discloses 2-piperazino-pteridine as well as its inhibiting effect on the phosphodiesterase of tumor cells and human thrombocytes in vitro. The pteridines described in it possess in the 4-position a dialkylamino, piperidino, morpholino, thiomorpholino or a 1-oxidothiomorpholino group.

Furthermore, in German published patent application DE 3445298, pteridines with a large number of different substituents in the 2-, 4-, 6- and 7-positions are described, wherein compounds with a 2-piperazino group on the pteridine structure are suitable as inhibitors for tumor growth and exhibit antithrombotic and metastasis-inhibiting characteristics.

In U.S. Pat. No. 2,940,972, tri- and tetra-substituted pteridine derivatives are disclosed, wherein general statements are made that these pteridines exhibit valuable pharmacological properties, namely coronary dilating, sedative, antipyretic and analgesic effects.

### BRIEF SUMMARY OF THE INVENTION

Consequently, an object of this invention is to provide new pteridine derivatives in a simple way, which exhibit further improved pharmacological properties in particular with regard to the inhibition of PDEs, e.g., for the prophylaxis and treatment of thrombo-embolic diseases, for the treatment of inflammatory, neurodegenerative and asthmatic diseases and the treatment of hemato-oncological diseases.

This object is solved according to the invention by a compound of the general formula (I) 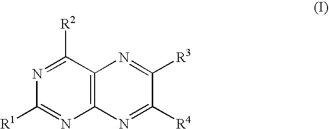
wherein:

R¹ signifies a piperazino, p-phenylenediamino, a 2,5-diazabicyclo-[2.2.1]-heptane, a 2,5-diazabicyclo-[2.2.2]-octane radical or a 3,8-diazabicyclo-[3.2.1]-octane radical, which in each case can be substituted with at least one substituent,

R², R⁴, which are in each case the same, signify a pyrrolidino, thiazolidino, oxazolidino or imidazolidino radical, which in each case can be substituted with at least one substituent, and

R³ signifies a halogen, alkyl, alkenyl, alkynyl, cycloalkyl, cycloalkenyl or aryl radical, which in each case can be substituted with at least one substituent, or a radical —X—R⁷,

wherein:

X signifies O, S or NR⁸,

R⁷ signifies an alkyl, alkenyl, alkynyl, cycloalkyl, cycloalkenyl or aryl radical, which in each case can be substituted with at least one substituent, and

R⁸ signifies hydrogen, an alkyl, alkenyl, alkynyl, cycloalkyl, cycloalkenyl or aryl radical, which in each case can be substituted with at least one substituent, and their acid addition salts, with the proviso that the compound of the general formula (I) is not 6-chloro-2-piperazino-4,7-dipyrrolidino-pteridine.

Furthermore, the object is solved by a compound of the general formula (I) 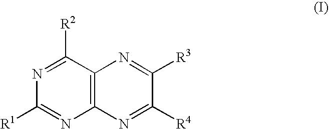
wherein:

R¹ signifies a piperazino, p-phenylenediamino, a 2,5-diazabicyclo-[2.2.1]-heptane- or a 2,5-diazabicyclo-[2.2.2]-octane radical, which in each case can be substituted with at least one substituent,

R², R⁴, which are in each case the same, signify a pyrrolidino, thiazolidino, oxazolidino or imidazolidino radical, which in each case can be substituted with at least one substituent, and

R³ signifies an alkyl, alkenyl, alkynyl, cycloalkyl, cycloalkenyl or aryl radical, which in each case can be substituted with at least one substituent, or a radical —X—R⁷,

wherein:

X signifies O, S or NR⁸,

R⁷ signifies an alkyl, alkenyl, alkynyl, cycloalkyl, cycloalkenyl or aryl radical, which in each case can be substituted with at least one substituent, and

R⁸ signifies hydrogen, an alkyl, alkenyl, alkynyl, cycloalkyl, cycloalkenyl or aryl radical, which in each case can be substituted with at least one substituent, and their acid addition salts.

Furthermore, the object is solved by a compound of the general formula (II), 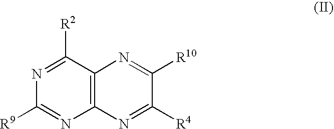
wherein:

R² and R⁴, which are in each case the same, signify a pyrrolidino, thiazolidino, oxazolidino or imidazolidino radical, which in each case can be substituted with at least one substituent, and

R⁹ and R¹⁰ are halogen,

with the proviso that the compound of the general formula (II) is not 2,6-dichloro-4,7-dipyrrolidino-pteridine.

Furthermore, the object is solved by a compound of the general formula (II), 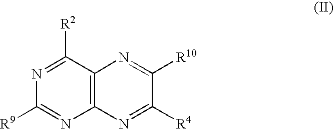
wherein:

R² and R⁴, which are in each case the same, signify a thiazolidino, oxazolidino or imidazolidino radical, which in each case can be substituted with at least one substituent, and

R⁹ and R¹⁰ are halogen.

The radical R¹ is preferably a piperazino radical.

The radicals R² or R⁴ are preferably pyrrolidino, thiazolidino, oxazolidino or imidazolidino radicals, in particular pyrrolidino or thiazolidino radicals.

The radical R³ is preferably a C₁-C₆-alkyl, C₂-C₆-alkenyl, C₂-C₆-alkynyl, C₃-C₆-cycloalkyl, C₃-C₆-cycloalkenyl or C₆-C₁₀-aryl radical. Furthermore, R³ is preferably a C₁-C₆-alkyl, C₁-C₆-alkoxy, C₁-C₆-alkylmercapto or a C₁-C₆-alkylamino radical. Especially preferably R³ is a C₁-C₃-alkylamino, C₁-C₃-alkoxy or C₁-C₃-alkylmercapto radical. Particularly R³ is a C₁-C₃-alkoxy or C₁-C₃-alkylmercapto radical, i.e., methoxy, ethoxy, propoxy, methylmercapto, ethylmercapto or propylmercapto, in particular methoxy or methylmercapto radical. If R³ is halogen, then fluorine, chlorine, bromine or iodine and especially chorine or bromine is preferred.

The radicals R¹ to R⁴ can be substituted with at least one, preferably one to three substituents, independently of one another.

R⁷ and R⁸ are preferably, independently of one another, a C₁-C₆-alkyl, C₂-C₆-alkenyl, C₂-C₆-alkynyl, C₃-C₆-cycloalkyl, C₃-C₆-cycloalkenyl or C₆-C₁₀-aryl radicals, in particular a C₁-C₃-alkyl radical.

In a preferred embodiment R⁸ is hydrogen or C₁-C₆-alkyl.

Furthermore, R⁹ and R¹⁰ are preferably, independently of one another, chlorine or bromine.

Examples of usual substituents include halogen, in particular Cl, F or Br, hydroxy, amino, nitro, CN, CF₃, C₁-C₄-alkyl, in particular C₁-C₃-alkyl, C₁-C₄-alkoxy, in particular C₁-C₃-alkoxy, C₁-C₄-alkylthio, C₃-C₇-Cycloalkyl, in particular C₃-C₆-cycloalkyl, C₃-C₆-cycloalkoxy, C₂-C₄-alkenyl, C₂-C₄-alkynyl, aryl, heteroaryl, NR⁵R⁶, COOR⁵, CONR⁵R⁶, NR⁵COR⁶, NR⁵COOR⁶, S(O)R⁵, SO₂R⁵, SO₂NR⁵R⁶, SO₃H,

and, provided with one or more substituents from this group, C₁-C₄-alkyl, C₁-C₄-alkoxy, C₁-C₄-alkylthio, C₃-C₇-cycloalkyl, C₂-C₄-alkenyl, C₂-C₄-alkynyl, aryl or heteroaryl,

wherein:

R⁵ and R⁶ signify independently of one another H, C₁-C₄-alkyl, aryl or heteroaryl or can form a C₃-C₇-cycloalkyl ring or C₃-C₇-cycloalkenyl ring and the ring can optionally contain one or more N, O and/or S atoms and/or a CH₂ group or several CH₂ groups can be replaced by one or more C═O groups.

The acid addition salts are usually pharmaceutically acceptable acid addition salts. Examples of these include organic and inorganic acid addition salts, such as hydrochloride, hydrobromide, phosphate, nitrate, perchlorate, sulphate, citrate, lactate, tartrate, maleate, fumarate, mandelate, benzoate, ascorbate, cinnamate, glycollate, methane sulphonate, formate, malonate, naphthalin-2-sulphonate, salicylate and acetate.

Furthermore, this invention relates to a method for the production of the above mentioned compounds, comprising the steps:

reacting 2,4,6,7-tetrachloropteridine with a compound, selected from the group consisting of pyrrolidine, thiazolidine, oxazolidine and imidazolidine;

reacting the product obtained with a compound selected from the group consisting of piperazine, p-phenylenediamine, 2,5-diazabicyclo[2.2.1]heptane, 2,5-diazabicyclo[2.2.2]octane and 3,8-diazabicyclo[3.2.1]octane;

reacting the product obtained with a compound selected from the group consisting of alkyl-M, alkenyl-M, alkynyl-M, cycloalkyl-M, cycloalkenyl-M, aryl-M, M—X—R⁷, or alkylformamide or dialkylformamide, in particular sodium alcoholate, sodium alkylthiolate or alkylformamide,

wherein:

R⁷signifies an alkyl, alkenyl, alkynyl, cycloalkyl, cycloalkenyl or aryl radical which in each case can be substituted with at least one substituent,

X signifies O, S or NR⁸,

M is Na or Li, and

R⁸ signifies hydrogen, an alkyl, alkenyl, alkynyl, cycloalkyl, cycloalkenyl or aryl radical which in each case can be substituted with at least one substituent.

The compounds of formula (I) or (II) according to the invention can here be produced by various methods and under usual reaction conditions.

The synthesis path for obtaining highly active PDE inhibitors is described in detail in Merz et al. 1998. Surprisingly, it has now been found that in positions 4 and 7, in the same way with a cyclic five-member amine which also can contain a further hetero atom, substituted pteridines display at least equally good or better inhibitors for PDE as the previously described compounds substituted differently at 4,7. This newly found, surprising property is a big advantage, because the production method for highly active PDE inhibitors is thereby significantly simplified. Consequently, the 4,7 disubstituted derivative can be produced in a simple manner and in a single step from 2,4,6,7-tetrachloropteridine, which can advantageously be used directly as a raw product. The substitutions at position 2 and then at position 6 occur in further reaction steps.

The starting materials used for the method according to the invention are either commercially available or can be produced from commercially available compounds according to known methods.

Furthermore, the object of this invention is solved by a pharmaceutical composition containing this compound and a pharmaceutically acceptable carrier.

In the following the pharmaceutical composition according to the invention, designated in the following also as the medicament, is explained in more detail.

The medicament according to the invention is administered primarily intravenously, but also in other types of application, such as intramuscularly, intra-arterially, intraperitoneally, intrathecally, subcutaneously, orally, perorally or also topically. Preferably, administration is by intravenous injection or intravenous infusion.

The medicament is produced according to known methods, wherein the compound according to the invention is used as such or, optionally, in combination with suitable pharmaceutical carrier substances. If the medicament according to the invention contains pharmaceutical carrier substances as well as the active substance, the content of active substance in this mixture is about 0.1 to 99.5, preferably about 0.5 to 95% by weight of the total mixture.

The medicament according to the invention can be applied in any suitable formulation with the prerequisite that the formation or maintenance of sufficient levels of active substance is ensured. This can, for example, be achieved by peroral or parenteral administration in suitable doses. Advantageously, the pharmaceutical preparation of the active substance is present in the form of standard doses which are matched to the required administered dosage. A standard dose may be, for example, a tablet, a coated tablet, a capsule, a suppository or a measured volume quantity of a powder, granulate, solution, emulsion or a suspension.

A “standard dose” in the sense of this invention is taken to mean a physically determined unit which contains an individual quantity of the active constituent in combination with a pharmaceutical carrier and its content of active substance corresponds to a fraction or multiple of a therapeutic single dose. A single dose preferably contains the quantity of active substance which is administered during an application and which normally corresponds to a whole, half, third or quarter of the daily dose. If only a fraction, such as half or quarter of the single dose is needed for a single therapeutically administered dose, then the standard dose is advantageously divisible, e.g. in the form of a tablet with a dividing groove.

The medicaments according to the invention can, if they are available in standard doses and intended for application, e.g., on persons, contain about 0.1 to 500 mg, preferably about 10 to 300 mg and particularly about 50 to 350 mg of active substance.

Generally in human medicine, the active substance(s) are administered in a daily dose of about 0.1 to 5, preferably about 1 to 3 mg/kg of body weight, where necessary in the form of a number, preferably about 1 to 3, of single intakes for achieving the desired results. A single intake contains the active substance(s) in quantities of about 0.1 to 10, preferably about 1 to 5 mg/kg of body weight. With oral treatment similar dosages can be applied.

The therapeutic administration of the medicament according to the invention can occur about 1 to 4 times daily at specified or varying time points, e.g. in each case before meals and/or in the evening. However, it may be necessary to deviate from the quoted dosages depending on the type, body weight and age of the individual to be treated, the type and severity of the disease, the type of preparation and the application of the medicaments as well as the time period or interval within which the administration occurs. Consequently, in some cases it may be sufficient to use less than the amount of active substance mentioned above, whereas in other cases the above listed quantities of active substance must be exceeded. It may also be practicable to administer the medicaments only once or at intervals of several days.

The specification of the necessary optimum dosage and type of application of the active substances can be made by any specialist based on his specialist knowledge.

The medicaments according to the invention normally comprise the compounds according to the invention and non-toxic, pharmaceutically compatible medication carriers, which as additive or dilution agents, are employed, for example, in solid, semi-solid or liquid form or as a means of enclosure, for example in the form of a capsule, a tablet coating, a bag or another receptacle for the therapeutically active constituent. A carrier material may, for example, act as an agent for the ingestion of the medicament by the body, as a formulation agent, sweetener, taste modifier, colorant or as preservative.

For peroral application, for example, tablets, coated tablets, capsules, for example of gelatine, dispersible powder, granulate, aqueous and oily suspensions, emulsions, solutions and syrups can be employed.

Tablets can contain inert filling agents, e.g., starches and starch derivatives, lactose, microcrystalline cellulose (MCC), cellulose and cellulose derivatives, calcium carbonate or sodium chloride; binding agents, e.g., starch, macrogols (PEGs), polyvidone (PVP), gelatine, alginates or arabine; lubricating agents, e.g., magnesium stearate, stearic acid, talcum or silicone oil; flow agents, e.g., highly dispersed silicon dioxide (aerosil); decomposition agents, e.g., starches and starch derivatives or crospovidone (qPVP); solubilizers; moisture retaining substances; gustatory correctors or colorants. They can also be provided with a coating or a jacket which can be of the type that it causes delayed release and resorption of the medicament in the gastro-intestinal tract, so that, for example, improved compatibility, assimilation or retardation is achieved.

Gelatine capsules may contain the pharmaceutical substance mixed with a solid, e.g., lactose or mannitol or an oily dilution agent e.g., olive, peanut or soya bean oil, apart from other carrier substances.

Aqueous suspensions can contain suspension agents, e.g., cellulose derivatives, sodium alginate, polyvidone, traganth rubber or arabine; dispersant or wetting agents, e.g., polyoxyethylene stearate, heptadeca-ethylene-oxycatanol, polyoxyethylene sorbitol-monooleate, or lecithin; preservatives, e.g. methyl- or propylhydroxy-benzoate; taste modifiers; sweeteners, e.g. saccharose, sodium cyclamate, dextrose or invert sugar syrup.

Oily suspensions may contain, for example, peanut, olive, sesame, coconut or paraffin oil and thickening agents, such as bees wax, high melting point wax or cetyl alcohol; also auxiliary substances such as emulsifying agents; sweeteners, taste modifiers; preservatives and antioxidants.

Powders and granulates dispersible in water may contain the compound according to the invention e.g. in a mixture with dispersing, wetting and suspension agents, e.g., those mentioned above as well as with sweeteners, taste modifiers and colorants.

Emulsions can, for example, contain olive, peanut or paraffin oil as well as emulsifying agents such as arabine, traganth rubber, phosphatides, sorbitan monooleate, polyoxyethylene sorbitan monooleate and sweeteners and taste modifiers as well as preservatives.

Aqueous solutions can contain preservatives, e.g., methyl- or propylhydroxybenzoates, thickening agents; taste modifiers; sweeteners, e.g., saccharose, sodium cyclamate, dextrose, invert sugar syrup as well as colorants.

For the parenteral application of pharmaceutical substances sterile injectable or infusable aqueous solutions, isotonic salt solutions or other solutions can be used. In addition, for example, sterile emulsions, suspensions or implants can be used, which can be of the type that a delayed release and resorption of the medicament preparation is caused, so that, for example, improved compatibility, assimilation or retardation is achieved.

The compound according to the invention in formula (I) can also be applied for the inhibition of cAMP-specific phosphodiesterases, for the inhibition of tumor growth, for the prophylaxis of thrombo-embolic diseases, as well as for the treatment of inflammatory, neurodegenerative and asthmatic diseases.

### BRIEF DESCRIPTION OF THE SEVERAL VIEWS OF THE DRAWINGS

The foregoing summary, as well as the following detailed description of the invention, will be better understood when read in conjunction with the appended drawing. For the purpose of illustrating the invention, there is shown in the drawing an embodiment which is presently preferred. It should be understood, however, that the invention is not limited to the precise arrangements and instrumentalities shown. In the drawings:

FIG. 1 is a representation of the IC₅₀ values of the growth inhibition when using the compounds E288, E289 and E499 on the human tumor cell lines COLO 205 and NCI-H460.

### DETAILED DESCRIPTION OF THE INVENTION

The following examples explain the invention.

1.) Production of 2,6-Dichloro-4,7-Dipyrrolidino-Pteridine

A solution of pyrrolidine (2.21 g; 31.1 mmol) and triethylamine (3.15 g; 31.1 mmol) in 50 mL of dioxane is added drop by drop at room temperature within 30 min to a suspension of 2,4,6,7-tetrachloropteridine (4 g; 14.8 mmol) in 100 mL of dioxane. The mixture is stirred for a further 0.5 hr and then the solvent is removed in a vacuum. The residue is washed with distilled water and dried over KOH. After flash chromatography on silica gel 60 (0.040-0.063 mm) with concentration of the flow agent (acetic ether/hexane 1:1) the product crystallises to bright yellow crystals. Yield>90% referred to pure 2,4,6,7-tetrachloropteridine.

2.) Production of 6-Chloro-2-Piperazino-4,7-Dipyrrolidino-Pteridine (E 499)

394 mg (1.16 mmol) of 2.6-dichloro-4,7-dipyrrolidino-pteridine and 400 mg (4.64 mmol) of piperazine are suspended in 20 mL of dioxane. The reaction mixture is heated for 1 hr under reflux and then the solvent removed in a vacuum. The residue is thoroughly washed with 30 mL of water, filtered and dried over KOH. Yellow solid, yield 90%.

3.) Production of 6-Methoxy-2-Piperazino-4,7-Dipyrrolidino-Pteridine (E 293)

A solution of 1 g of sodium in 10 mL of methanol is added to a suspension of 200 mg of E 499 in 50 mL of dioxane. The mixture is heated under reflux with stirring for 2 hr. The solvent is largely removed on the rotary evaporator, the residue taken up in 50 mL of water and the separated raw product is filtered off. After flash chromatography (EtOH+2.5% triethylamine) the end product is obtained as a pale yellow solid. Yield 76%.

4.) Production of 2,6-Dichloro-4,7-Dithiazolidino-Pteridine

A solution of 2.73 g (30.6 mmol) of thiazolidine and 3.09 g (30.6 mmol) of triethylamine in 50 mL of dioxane is added drop by drop to a suspension of 2,4,6,7-tetrachloropteridine (3.93 g; 14.6 mmol) in 100 mL of dioxane. The solvent is removed in a vacuum, the residue washed with water and dried. After flash chromatography on silica gel 60 (0.040-0.063 mm) the product crystallizes out of the flow agent (acetic ether/hexane 1:2). Bright yellow needles, yield>90% referred to pure 2,4,6,7-tetrachloropteridine.

5.) Production of 6-Chloro-2-Piperazino-4,7-Dithiazolidino-Pteridine (E 288)

2,6-dichloro4,7-dithiazolidino-pteridine (644 mg; 1.72 mmol) and piperazine (166 mg; 1.93 mmol) are suspended in 25 mL of dioxane. Triethylamine (195 mg; 1.93 mmol) is added to it and the mixture heated for 5 hr under reflux. Then the solvent is removed in a vacuum, the residue washed thoroughly with water and dried. After flash chromatography a luminous yellow solid is obtained. Yield 80%.

6.) Production of 6-Methoxy-2-Piperazino-4,7-Dithiazolidino-Pteridine (E 289)

A solution of 800 mg of sodium in 8 mL of methanol is added drop by drop to a suspension of 6-chloro-2-piperazino-4,7-dithiazolidino-pteridine (158 mg; 0.037 mmol) in 30 mL of dioxane and the mixture is heated for 2 hr under reflux. The solvent is removed on the rotary evaporator, the residue taken up in 40 mL of water and the precipitated raw product filtered off. After flash chromatography the end product is obtained as a beige-coloured solid. Yield 75%.

7.) Production of 6-Methylthio-2-Piperazino-4,7-Dipyrrolidino-Pteridine (E 294)

6-chloro-2-piperazino4,7-dipyrrolidino-pteridine (500 mg; 1.29 mmol) and sodium methane-thiolate (133 mg; 1.9 mmol) are suspended in 15 mL of hexamethyl phosphoric acid triamide and heated to 80° C. for 1.5 hr. The reaction mixture, once cooled down, is mixed with 50 mL of water, the precipitate filtered off and washed with water. The filtrate is extracted 3 times with 75 mL of chloroform each time. The chloroform phases are combined, dried with magnesium sulphate, dried by centrifuging and combined with filtered precipitate. After flash chromatography on silica gel (flow agent: ethanol+5% triethylamine) the flow agent is centrifuged off, the residue thoroughly washed with water, dissolved in 0.1 N HCl and precipitated out with 5% ammonia solution. Bright yellow solid. Yield 55%.

8.) Proliferation Assay

The inhibition of the growth of tumor cells due to the compounds according to the invention was determined on the human cell line LXFL529L. As a proliferation assay the sulforhodamin B assay as described by Skehan et al. (J. Natl. Cancer Inst. 82:1107-1112 (1990)) was used.

The following are examples of the IC₅₀ values [μM]:

| | |
|---|---|
| 6-methoxy-2-piperazino-4,7-dipyrrolidino-pteridine: | 3.4 ± 1.0 |
| 6-methylthio-2-piperazino-4,7-dipyrrolidino-pteridine: | 3.0 ± 0.3 |
| 6-chloro-2-piperazino-4,7-dipyrrolidino-pteridine: | 4,7 ± 0.4 |

Further in-vitro results of the compounds according to the invention E288 (6-chloro-2-piperazino-4,7-dithiazolidino-pteridine) and E289 (6-methoxy-2-piperazino-4,7-dithiazolidino-pteridine) are summarized in the following in comparison to E499 (6-chloro-2-piperazino-4,7-dipyrrolidino-pteridine) (all IC₅₀ figures are described in [μM]).

| | E288 | E289 | E499 |
|---|---|---|---|
| COLO 205 | 6.19 | 9.64 | 9.75 |
| NCI-H460 | 18.2 | 13.9 | 31.6 |

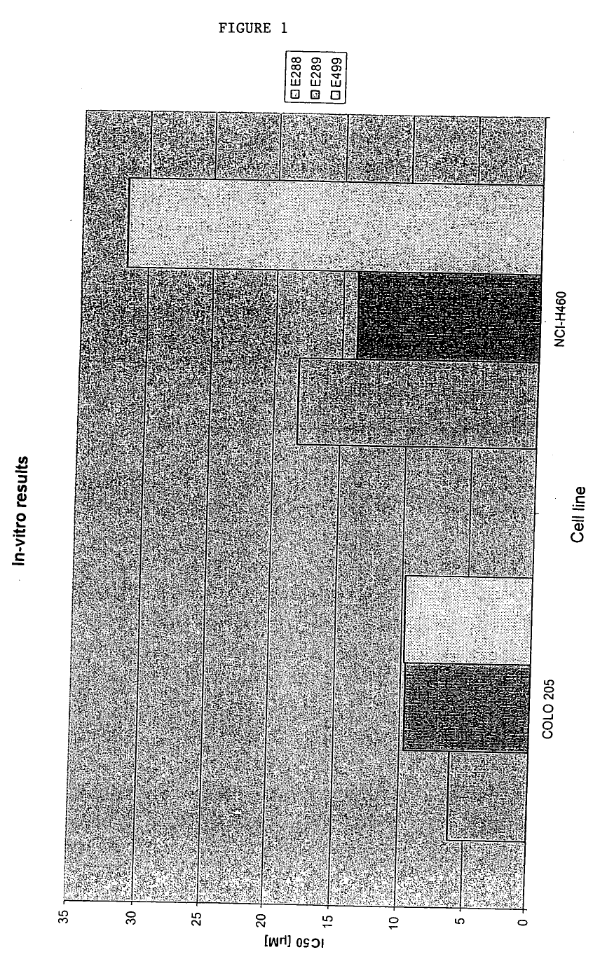

The anti-tumor spectrum of the new active substances is wide, because in addition to the large-cell lung carcinoma LXFL529, as well as the colon carcinoma COLO 205 and bronchial carcinoma NCI-H460 (FIG. 1/1), other tumor cells also prove to be sensitive in the XTT assay (Scudiero et al., Cancer Res. 48:4827-4833 (1988)) with IC₅₀ values in the lower micromolar range. This includes the human cell lines A431 (fibroblasts), OVCAR-3 (ovarian carcinoma), BT-549 and MCF-7 (mammary carcinoma), SK-MEL-28 and SK-MEL-5 (melanoma), SW 620 and HCT-15 (colon), A549 (lung carcinoma) as well as the glioblastoma cell line C6 of the rat.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A compound of the general formula (I) 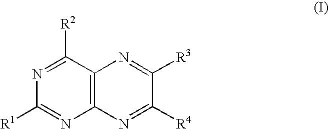
wherein:
R¹ signifies a piperazino, p-phenylenediamino, a 2,5-diazabicyclo-[2.2.1]-heptane, a 2,5-diazabicyclo-[2.2.2]-octane radical or a 3,8-diazabicyclo-[3.2.1]-octane radical, which in each case can be substituted with at least one substituent,
R², R⁴, which are in each case the same, signify a pyrrolidino, thiazolidino, oxazolidino or imidazolidino radical, which in each case can be substituted with at least one substituent, and
R³ signifies a halogen, alkyl, alkenyl, alkynyl, cycloalkyl, cycloalkenyl or aryl radical, which in each case can be substituted with at least one substituent, or a radical —X—R⁷,
wherein:
X signifies O, S or NR⁸,
R⁷ signifies an alkyl, alkenyl, alkynyl, cycloalkyl, cycloalkenyl or aryl radical, which in each case can be substituted with at least one substituent, and
R⁸ signifies hydrogen, an alkyl, alkenyl, alkynyl, cycloalkyl, cycloalkenyl or aryl radical, which in each case can be substituted with at least one substituent,
and their acid addition salts,
with the proviso that the compound of the general formula (I) is not 6-chloro-2-piperazino-4,7-dipyrrolidino-pteridine.

2. A compound of the general formula (I) 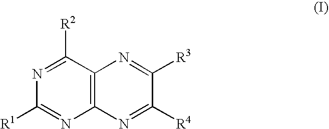
wherein:
R¹ signifies a piperazino, p-phenylenediamino, a 2,5-diazabicyclo-[2.2.1]-heptane or a 2,5-diazabicyclo-[2.2.2]-octane radical, which in each case can be substituted with at least one substituent,
R², R⁴, which are in each case the same, signify a pyrrolidino, thiazolidino, oxazolidino or imidazolidino radical, which in each case can be substituted with at least one substituent, and
R³ signifies an alkyl, alkenyl, alkynyl, cycloalkyl, cycloalkenyl or aryl radical, which in each case can be substituted with at least one substituent, or a radical —X—R⁷,
wherein:
X signifies O, S or NR⁸,
R⁷ signifies an alkyl, alkenyl, alkynyl, cycloalkyl, cycloalkenyl or aryl radical, which in each case can be substituted with at least one substituent, and
R⁸ signifies hydrogen, an alkyl, alkenyl, alkynyl, cycloalkyl, cycloalkenyl or aryl radical, which in each case can be substituted with at least one substituent,
and their acid addition salts.

3. The compound according to claim 1, wherein R¹ signifies a piperazino radical.

4. The compound according to claim 2, wherein R¹ signifies a piperazino radical.

5. The compound according to claim 1, wherein R² and R⁴ signify a thiazolidino, oxazolidino or imidazolidino radical.

6. The compound according to claim 2, wherein R² and R⁴ signify a thiazolidino, oxazolidino or imidazolidino radical.

7. The compound according to claim 1, wherein R³ signifies a C₁-C₃-alkyl, C₁-C₃-alkoxy, C₁-C₃-alkylmercapto or a C₁-C₃-alkylamino radical.

8. The compound according to claim 2, wherein R³ signifies a C₁-C₃-alkyl, C₁-C₃-alkoxy, C₁-C₃-alkylmercapto or a C₁-C₃-alkylamino radical.

9. The compound according to claim 1, wherein R³ signifies methoxy, ethoxy, propoxy, methylmercapto, ethylmercapto or propylmercapto.

10. The compound according to claim 2, wherein R³ signifies methoxy, ethoxy, propoxy, methylmercapto, ethylmercapto or propylmercapto.

11. The compound according to claim 1, wherein R³ signifies chlorine or bromine.

12. The compound according to claim 1, wherein the acid addition salts represent physiologically compatible acid addition salts of inorganic or organic acids.

13. The compound according to claim 2, wherein the acid addition salts represent physiologically compatible acid addition salts of inorganic or organic acids.

14. A compound of the general formula (II), 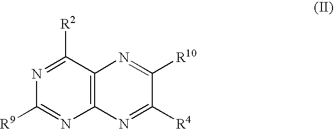
wherein:
R², R⁴, which are in each case the same, signify a pyrrolidino, thiazolidino, oxazolidino or imidazolidino radical, which in each case can be substituted with at least one substituent, and
R⁹ and R¹⁰ are halogen,
with the proviso that the compound of the general formula (II) is not 2,6-dichloro-4,7-dipyrrolidino-pteridine.

15. A compound of the general formula (II), 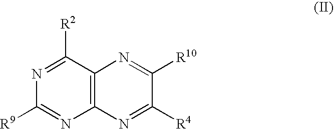
wherein:
R² and R⁴, which are in each case the same, signify a thiazolidino, oxazolidino or imidazolidino radical, which in each case can be substituted with at least one substituent and
R⁹ and R¹⁰ are halogen.

16. A pharmaceutical composition, containing the compound according to claim 1 and at least one pharmaceutically acceptable carrier.

17. A pharmaceutical composition, containing the compound according to claim 2 and at least one pharmaceutically acceptable carrier.

18. A pharmaceutical composition, containing the compound according to claim 14 and at least one pharmaceutically acceptable carrier.

19. A pharmaceutical composition, containing the compound according to claim 15 and at least one pharmaceutically acceptable carrier.

20. A method of inhibiting cAMP-specific phosphodiesterases, comprising applying a compound according to claim 1.

21. A method of inhibiting cAMP-specific phosphodiesterases, comprising applying a compound according to claim 2.

22. A method of inhibiting cAMP-specific phosphodiesterases, comprising applying a compound according to claim 14.

23. A method of inhibiting cAMP-specific phosphodiesterases, comprising applying a compound according to claim 15.

24. A method for treatment and/or prophylaxis of a disease selected from the group consisting of hemato-oncological diseases, neurodegenerative diseases, inflammatory diseases, thrombo-embolic diseases, and asthmatic diseases, comprising applying a compound according to claim 1.

25. A method for treatment and/or prophylaxis of a disease selected from the group consisting of hemato-oncological diseases, neurodegenerative diseases, inflammatory diseases, thrombo-embolic diseases, and asthmatic diseases, comprising applying a compound according to claim 2.

26. A method for treatment and/or prophylaxis of a disease selected from the group consisting of hemato-oncological diseases, neurodegenerative diseases, inflammatory diseases, thrombo-embolic diseases, and asthmatic diseases, comprising applying a compound according to claim 14.

27. A method for treatment and/or prophylaxis of a disease selected from the group consisting of hemato-oncological diseases, neurodegenerative diseases, inflammatory diseases, thrombo-embolic diseases, and asthmatic diseases, comprising applying a compound according to claim 15.

28. A method for producing a compound according to claim 1, comprising the steps:
reacting 2,4,6,7-tetrachloropteridine with a compound, selected from the group consisting of pyrrolidine, thiazolidine, oxazolidine and imidazolidine;
reacting the product obtained with a compound selected from the group consisting of piperazine, p-phenylenediamine, 2,5-diazabicyclo[2.2.1]heptane, 2,5-diazabicyclo[2.2.2]octane and 3,8-diazabicyclo[3.2.1]octane; and
reacting the product obtained with a compound selected from the group consisting of alkyl-M, alkenyl-M, alkynyl-M, cycloalkyl-M, cycloalkenyl-M, aryl-M, M—X—R⁷, alkylformamide, dialkylformamide, in particular sodium alcoholate, sodium alkylthiolate and alkylformamide,
wherein:
R⁷ signifies an alkyl, alkenyl, alkynyl, cycloalkyl, cycloalkenyl or aryl radical which in each case can be substituted with at least one substituent,
X signifies O, S or NR⁸,
M is Na or Li, and
R⁸ signifies hydrogen, an alkyl, alkenyl, alkynyl, cycloalkyl, cycloalkenyl or aryl radical which in each case can be substituted with at least one substituent.
